(19) United States
(12) Patent Application Publication
(10) Pub. No.: US 2005/0189302 A1
(43) Pub. Date: Sep. 1, 2005

(54) Viral inactivation using ozone

(76) Inventors: Joseph S. Latino, Brooklyn, NY (US); Steven A. Keyser, Salt Lake City, UT (US)

Correspondence Address: John M. Guynn;WORKMAN NYDEGGER, 1000 Eagle Gate Tower, 60 East South Temple, Salt Lake City, UT, 84111, US

(21) Appl. No.: 10/963,139

(22) Filed: Oct. 11, 2004

Related U.S. Application Data
(63) Continuation-in-part of application No. 10/910,485, filed on Aug. 2, 2004.
(63) Continuation-in-part of application No. 10/910,439, filed on Aug. 2, 2004.
(60) Provisional application No. 60/553,774, filed on Mar. 17, 2004.
(60) Provisional application No. 60/491,997, filed on Jul. 31, 2003.

(51) Int. Cl. C02F 1/00

(52) U.S. Cl. 210/739

(57) ABSTRACT

A method to inactivate viruses in a biological fluid and a non-fluid target to produce a non-infectious biological fluid or non-fluid target. The method involves subjecting an amount of a fluid or a target containing a virus including lipid-enveloped viruses, to an amount of ozone delivered by an ozone delivery system. The method may provide for maintaining the biological integrity of the biological fluid or the non-fluid target. All gas contacting surfaces of the system, including one or more gas-fluid contact devices are made from ozone-inert construction.

### CROSS-REFERENCE TO RELATED APPLICATIONS

This application is a continuation-in-part of copending U.S. application Ser. No. 10/910,485, filed Aug. 2, 2004, and also a continuation-in-part of copending U.S. application Ser. No. 10/910,439, filed Aug. 2, 2004, both of which claim the benefit of earlier-filed U.S. provisional application Ser. No. 60/553,774, filed Mar. 17, 2004, and U.S. provisional application Ser. No. 60/491,997, filed Jul. 31, 2003. The disclosures of the foregoing applications are incorporated herein in their entirety.

### BACKGROUND OF THE INVENTION

Historically, ozone has been used as a disinfectant or sterilizing agent in a wide variety of applications. These include fluid-based technologies such as: purification of potable water, sterilization of fluids in the semi-conductor industry, disinfection of wastewater and sewage, and inactivation of pathogens in biological fluids. Ozone has also been used in the past as a topical medicinal treatment, as a systemic therapeutic and as a treatment of various fluids that were subsequently used to treat a variety of diseases. Specifically, there have been numerous attempts utilizing a variety of technologies to inactivate viruses in biological fluids.

Previous technologies were incapable of measuring and differentiating between the amount of ozone that was delivered and the amount of ozone actually absorbed and utilized. This meant previous medicinal technologies for use in patients were incapable of measuring, reporting or differentiating the amount of ozone delivered from the amount that was actually absorbed and utilized. This problem made regulatory approval as a therapeutic unlikely. In the treatment of virally contaminated fluids, previous technologies were also incapable of measuring, reporting or differentiating the amount of ozone delivered from the amount that was actually absorbed by the fluid and utilized in the inactivation of viruses. Furthermore, any technology considered to inactivate viruses with ozone must be able to maintain the biological integrity of the fluid for its subsequent intended use.

In addition, early approaches of mixing ozone with fluids employed gas-fluid contacting devices that were engineered with poor mass transfer efficiency of gas to fluids. Later, more efficient gas-fluid contacting devices were developed, but these devices used construction materials that were not ozone inert and therefore, reacted and absorbed ozone. This resulted in absorption of ozone by the construction materials making it impossible to determine the amount of ozone delivered to and absorbed by the fluid. Furthermore, ozone absorption by construction materials likely caused oxidation and the subsequent release of contaminants or deleterious byproducts of oxidation into the fluid.

Experimental research confirms the problem of ozone absorption by construction materials. An ozone/oxygen admixture at 1200 ppmv was passaged through a commercially available membrane oxygenator. For a period in excess of two hours, a majority of the ozone delivered to the device was absorbed by the construction materials. This data strongly suggests commercially available membrane gas-fluid contacting devices, made from ozone reactive materials, cannot be used with ozone, and supports the necessity to develop novel ozone-inert gas-fluid contacting devices.

In addition, prior methods do not quantify the amount of ozone that does not react with the biological fluid. The inability to measure residual-ozone has led to inaccurate and imprecise determinations of both the amount of ozone delivered to the fluid, and the amount of ozone actually absorbed and utilized by the fluid. Prior techniques also failed to recognize that fluids of varying composition display different absorption phenomena.

### SUMMARY OF THE INVENTION

A method of inactivating viruses in a biological fluid or non-fluid target to produce a non-infectious biological fluid or target is provided. The method involves subjecting an amount of a fluid or a target containing a virus including lipid-enveloped viruses, to an amount of ozone delivered by an ozone delivery system. The method may provide for maintaining the biological integrity of the biological fluid or target. The method employs an ozone-delivery system for delivering and manufacturing a measured amount of an ozone/oxygen admixture, which is able to measure, control and report and differentiate between delivered-ozone and the absorbed-dose of ozone. The system may include improved gas-fluid contacting devices that maximize gas-fluid mass transfer. All gas contact surfaces of the system, including one or more gas-fluid contact devices are made from ozone-inert construction materials that generally do not absorb ozone or introduce contaminants or deleterious byproducts of oxidation into a fluid.

### BRIEF DESCRIPTION OF DRAWINGS

To further clarify the above, a more particular description of an ozone delivery system will be rendered by reference to specific embodiments thereof that are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1 illustrates a schematic diagram of the ozone delivery system described in the present invention.

FIG. 2 illustrates a schematic diagram of a sphere-containing gas-fluid contact device.

FIG. 3 illustrates a schematic diagram of a variable pitch gas-fluid contacting device.

FIG. 4 illustrates a schematic diagram of a variable pitch platform used in conjunction with FIG. 3.

FIG. 5 illustrates a schematic diagram of a continuous loop configuration for CD z M; fluid flow in a dialysis-like format.

FIG. 6 illustrates a schematic diagram of a non-fluid topical application.

### DETAILED DESCRIPTION OF THE INVENTION

The method of inactivation of viruses in biological fluids generates byproducts of ozonation, including lipid peroxides that are a result of the absorption of an absorbed-dose of ozone from the delivered-ozone by the fluid. The lipid peroxides are quantifiable by assay for lipid peroxides. A direct relationship can be derived between the absorbed-dose of ozone absorbed by a biological fluid and these quantifiable lipid peroxides. The lipid peroxides generated are pharmacologically active and possess intrinsic antiviral activity. A quantifiable pharmacologically antiviral amount of lipid peroxides generated when a biological fluid has absorbed an absorbed-dose of ozone is considered a drug-blood product.

Definitions

An ozone/oxygen admixture refers to a concentration of ozone in an oxygen carrier gas. Various units of concentration utilized by those skilled in the art include: micrograms of ozone per milliliter of oxygen, parts (ozone) per million (oxygen) by weight (‘ppm’) and parts per million by volume (‘ppmv’). As a unit of concentration for ozone in oxygen, ppmv is defined as the molar ratio between ozone and oxygen. One ppmv ozone is equal to 0.00214 micrograms of ozone per milliliter of oxygen. Additionally, one ppm ozone equals 0.00143 micrograms of ozone per milliliter of oxygen. In terms of percentage ozone by weight, 1% ozone equals 14.3 micrograms of ozone per milliliter of oxygen. All units of concentration and their equivalents are calculated at standard temperature and pressure (i.e. 25° C. at 1 atmosphere).

Delivered-ozone is the amount of ozone contained within a volume of an ozone/oxygen admixture that is delivered to a fluid.

Absorbed-ozone is the amount of delivered-ozone that is actually absorbed and utilized by a measured amount of fluid.

Residual-ozone is the amount of delivered-ozone that is not absorbed such that:
Residual-ozone=delivered-ozone−absorbed-dose of ozone

An interface is defined as the contact between a fluid and an ozone/oxygen admixture.

Interface-time is the time that a fluid resides within a gas-fluid contacting device and is interfaced with an ozone/oxygen admixture.

Interface surface area is defined as the dimensions of the surface within a gas-fluid contacting device over which a fluid flows and contacts an ozone/oxygen admixture.

Elapsed-time is the time that a fluid circulates throughout an ozone delivery system, including passage through one or more gas-fluid contacting devices, connecting tubing and an optional reservoir.

Ozone-inert materials are defined as construction materials that do not react with ozone in a manner that introduces contaminants or deleterious byproducts of oxidation of the construction materials into a fluid.

Non-reactive is defined as not readily interacting with other elements or compounds to form new chemical compounds.

Measured-data is defined as information collected from various measuring components (such as an inlet ozone concentration monitor, exit ozone concentration monitor, gas flow meter, fluid pump, data acquisition device, humidity sensor, temperature sensor, pressure sensor, absorbed oxygen sensor) throughout the system.

Calculated-data is defined as the mathematical treatment of measured-data by a data acquisition device.

Absorption of ozone by a biological fluid is defined as the phenomenon wherein ozone reacts with the fluid by a variety of mechanisms, including oxidation. Regardless of the mechanism(s) involved, the reaction occurs instantaneously, and the products of this reaction include oxidative products, of which lipid peroxides are an example.

A biological fluid is defined as a composition originating from a biological organism of any type. Examples of biological fluids include: blood, blood products and other fluids such as saliva, urine, feces, semen, milk, tissue, tissue samples, homogenized tissue samples, gelatin, and any other substance having its origin in a biological organism. Biological fluids may also include synthetic materials incorporating a substance having its origin in a biological organism, such as a vaccine preparation containing alum and a virus (the virus being the substance having its origin in a biological organism), cell culture media, cell cultures, viral cultures, and other cultures derived from a biological organism.

A blood product is defined as including blood fractionates and therapeutic protein compositions containing proteins derived from blood. Fluids containing biologically active proteins other than those derived from blood may also be treated by the method.

In vivo use of a material or compound is defined as the introduction of a material or compound into a living human, mammal, or vertebrate.

In vitro use of a material or compound is defined as the use of the material or compound outside a living human, mammal, or vertebrate, where neither the material nor compound is intended for re-introduction into a living human, mammal, or vertebrate. An example of an in vitro use would be the analysis of a component of a blood sample using laboratory equipment.

Ex vivo use of a process is defined as using a process for treatment of a biological material such as a blood product outside of a living human, mammal, or vertebrate. For example, removing blood from a human and subjecting that blood to a method to inactivate viruses is defined as an ex vivo use of that method if the blood is intended for reintroduction into that human or another human. Reintroduction of the human blood into that human or another human would be an in vivo use of the blood, as opposed to an ex vivo use of the method.

Synthetic media is defined as an aqueous synthetic blood or blood product storage media.

A pharmaceutically acceptable carrier or pharmaceutically acceptable vehicle is defined as any liquid including: water, saline, a gel, salve, solvent, diluent, fluid ointment base, liposome, micelle, and giant micelles, which is suitable for use in contact with a living animal or human tissue without causing adverse physiological responses, and which does not interact with the other components of the composition in a deleterious manner.

A virus is defined as any of various simple submicroscopic parasites of plants, animals, and bacteria that often cause disease and that consists essentially of a core of RNA or DNA, either single stranded or double stranded, and surrounded by a protein coat. Viruses can be further differentiated based upon the presence or absence of a lipid envelope surrounding the protein coat. Viruses are typically not considered living organisms as they are unable to replicate without a host cell.

Biologically active is defined as capable of effecting a change in the living organism or component thereof. Biologically active with respect to biologically active protein as referred to herein does not refer to proteins that are part of the microorganisms being inactivated.

A non-infectious biological fluid is defined as being unable to cause one or more diseases.

The biological integrity of a biological fluid is a fluid that subsequent to the application of the inactivation method described herein, sufficiently maintains its replacement value for intended use in vivo or in vitro.

A drug-blood product is defined as a biological fluid that contains a quantifiable pharmacologically antiviral amount of lipid peroxides generated from the absorption of an absorbed-dose of ozone according to the method described herein, by the biological fluid.

Biologic Products

The use of ozone for viral inactivation in biological fluids is of particular interest as the safety of the blood supply is an issue of universal concern. While transfusion associated viral infections have been considerably reduced by testing, transmission of human immunodeficiency virus (HIV), hepatitis B virus (HBV) and hepatitis C virus (HCV) continue to occur in 1/450,000 to 600,000 units, 1/200,000 units and 1/3000 units. Testing is not an option for some viruses. Cytomegalovirus is commonly found within the blood supply yet is of clinical importance only to immunocompromised patients for which infection can be fatal. Universal screening for CMV would lead to serious reduction in eligible donors and thus a reduction in the national blood supply. Special donor pools must be used for these patients at present. It is also recognized that other unknown viruses or new strains of known viruses may enter into the blood supply and will not be identified until transfusion-related death and disease is noted, nor will they be able to be screened for until tests become available. The identification of hepatitis G in blood units is a recent example of such an occurrence.

Examples of materials to be treated to render them non-infectious include: whole blood and aqueous compositions containing biologically active proteins derived from blood or blood fractionates. Packed red cells, platelets and plasma (both fresh and fresh frozen plasma) include such fractionates. In addition, therapeutic protein compositions containing proteins derived from blood, such as fluids containing biologically active proteins useful in the treatment of medical disorders, e.g. factor VIII, Von Willebrand factor, factor IX, factor X, factor XI, Hageman factor, prothrombin, antithrombin III, fibronectin, plasminogen, plasma protein fraction, immune serum globulin, modified immune globulin, albumin, plasma growth hormones, somatomedin, plasminogen-streptokinase complex, ceruloplasmin, transferrin, haptoglobin, antitrypsin and prekallikrein may be treated by one embodiment of the inactivation method. Other fluids, which may benefit from similar treatment include peritoneal solutions used for peritoneal dialysis as they can be contaminated during connection, leading to peritoneal infections. In addition, gelatin, gelatin-containing products, cosmetic formulations, pharmaceutical and dietary supplements, and any other animal derived products are included.

Solvent-detergent methods of blood component decontamination dissolve phospholipid membranes surrounding viruses such as HIV, and do not damage protein components of blood. This process is a standard process employed for both plasma and plasma-derived protein fractionates but is not compatible with cellular components (red blood cells or platelets) due to extensive damage to cellular membranes. Methods of inactivation of viruses in both plasma and cellular components presently being investigated include targeted chemotherapeutics (e.g. frangible anchor-linked-effectors), photochemotherapeutics (e.g. psoralen derivatives), gamma-irradiation and photodynamic antimicrobial chemotherapy (e.g. thiazine dyes such as thionin, azure A, azure B, azure C, methylene blue and Toluidine blue, and riboflavin).

In one embodiment a method of inactivating viruses in biological fluids while maintaining the biological integrity of the fluid is described.

In one embodiment, the biological fluid is a blood component. In another embodiment, the blood product is either plasma, red blood cell preparations or platelet concentrates. In one example a method of inactivation is performed in a blood bank or similar setting, wherein the biological fluid is passed through a gas-fluid contacting device and subjected to the method described herein. The blood product is then suitable for intended in vitro or in vivo use.

Biological fluids are generally aqueous in nature and in many cases are in a fluid state compatible with treatment by the method described herein. However, in those instances where required (i.e. blood and blood fractionates), a compatible aqueous buffered carrier may be added to lower viscosity, anticoagulate, or to otherwise permit the method to occur more effectively. In addition, in one method lipid peroxides are reproducibly generated as a reactive oxidative intermediate byproduct of the process, in a carrier fluid to be subsequently added to a biological fluid.

Platelet additive solutions may contain physiological saline solution, buffers, and other components including magnesium chloride and sodium gluconate. These solutions are useful carriers for platelet concentrates and allow for maintenance of cell quality and metabolism during storage, reduce plasma content and extend storage life. The pH of such solutions is preferably between about 7.0 and 8.0.

The described methods inactivate viruses present in biological fluids through a single process. In addition, the methods may be used to inactivate viruses in conjunction with other viral inactivation processes either prior, subsequent or contemporaneously with the methods instructed herein.

Inactivation of Potential Viruses

A virus is defined as any of various simple submicroscopic parasites of plants, animals, and bacteria that often cause disease and that consists essentially of a core of RNA or DNA, either single stranded or double stranded, and surrounded by a protein coat. Viruses can be further differentiated based upon the presence or absence of a lipid envelope surrounding the protein coat. Viruses are typically not considered living organisms as they are unable to replicate without a host cell.

In the case of viral inactivation methods for material to be used by humans, whether in vivo or in vitro, the detection method can theoretically be defined as the measurement of the level of infection, with disease as the result of exposure to the material. The threshold for infection below which the inactivation method is complete is then taken to be the level of inactivation, which is sufficient to prevent the disease from occurring due to contact with the material. It is recognized that in this practical scenario, it is not essential that the methods result in “total inactivation”. That is to say, “substantial inactivation” will be adequate as long as the treated material is insufficient to cause disease, or rendered “non-infectious”. The inactivation methods render the viruses substantially inactivated. In one embodiment, the inactivation method renders the viruses in blood preparations inactivated and it is inferred to be substantially inactivated, or non-infectious, as defined above.

In one embodiment, the inactivation method provides an ex vivo method of inactivating viruses, including lipid-enveloped viruses, in biological fluids prior to use in vivo or in vitro. Infectious viruses, which may be inactivated by the method include those containing a degree of lipid in their viral envelope. In another embodiment, the method reduces infectivity of infectious viruses and may also provide vaccines against these viruses.

A target list of viruses, which are susceptible to inactivation by the method described is in Table 1 below. This list is not exhaustive, and is merely representative of the variety of viruses ozone can inactivate. In one embodiment, the method is particularly suited for inactivating lipid-enveloped viruses, such as HIV, hepatitis B and hepatitis C.

TABLE 1
Viruses Inactivated by Ozone

| Family | Virus |
|---|---|
| Arena | Pichinde |
| | Lassa |
| Bunya | Turlock |
| | California encephalitits |
| Corona | Coronaviruses |
| Hepadna | hepatitis B |
| Herpes | Herpes simplex 1 |
| | Herpes simplex 2 |
| | Pseudorabies |
| Cytomegalovirus | |
| Orothomyxo | Influenza |
| Paramyxo | Measles |
| | Mumps |
| | Parainfluenza 2 and 3 |
| Pox | Vaccinia |
| | Fowl Pox |
| Retro | HIV |
| | Avian sarcoma |
| | Murine sarcoma |
| | Murine leukemia |
| Rhabdo | Vesicular stomatitis virus |
| Toga | Western equine encephalitis |
| | Dengue 2 |
| | Dengue 4 |
| | St. Louis encephalitis |
| Bacteriophage | Lambda |
| | T2 |
| Rickettsia | R. akari (rickettsialpox) |

In addition, a more comprehensive list of viral infectious organisms that may be inactivated by the method described herein including the lipid-containing viruses of the following genuses: Alphavirus (alphaviruses), Rubivirus (rubella virus), Flavivirus (Flaviviruses), Pestivirus (mucosal disease viruses), unnamed hepatitis C virus, Torovirus, (toroviruses), Arterivirus, (arteriviruses), Rubulavirus (rubulavriuses), Morbillivirus (morbilliviruses), Pneumovirinae (pneumoviruses), Vesiculovirus (vesiculoviruses), Lyssavirus (lyssaviruses), Ephemerovirus (ephemeroviruses), Cytorhabdovirus (plant rhabdovirus group A), Nucleorhabdovirus (plant rhabdovirus group B), Filovirus (filoviruses), unnamed Thogoto-like viruses, Phlebovirus (phleboviruses), Nairovirus (nairoviruses), Hantavirus (hantaviruses), Tospovirus (tospoviruses), unnamed mammalian type B retroviruses, unnamed mammalian and reptilian type C retroviruses, unnamed type D retroviruses, Lentivirus (lentiviruses), Spumavirus (spumaviruses), Orthohepadnavirus (hepadnaviruses of mammals), Avihepadnavirus (hepadnaviruses of birds), Simplexvirus (simplexviruses), Varicellovirus (varicelloviruses), Betaherpesvirinae, Cytomegalovirus (cytomegaloviruses), Muromegalovirus (murine cytomegaloviruses), Roseolovirus (human herpes virus 6), Gammaherpesvirinae (lymphocyte-associated herpes viruses), Lymphocryptovirus (Epstein-Barr-like viruses), Rhabdinovirus (saimiriateles-like herpes viruses), Orthopoxvirus (orthopoxviruses), Parapoxvirus (parapoxviruses), Avipoxvirus (fowlpox viruses), Capripoxvirus (sheeppox-like viruses), Leporipoxvirus (rnyxomaviruses), Suipoxvirus (swine-pox viruses), Molluscipoxvirus (molluscum contagiosum viruses), Yatapoxvirus (yatapox and tanapox viruses), unnamed African swine fever-like viruses, Iridovirus (small iridescent insect viruses), Ranavirus (iridoviruses), Lymphocystivirus (lymphocystis viruses of fish), Togaviridae, Flaviviridae, Enabdoviridae, and any other lipid-containing virus.

These viruses include the following human and animal viruses: Ross River virus, fever virus, dengue viruses, Murray Valley encephalitis virus, tick-borne encephalitis viruses (including European and far eastern tick-borne encephalitis viruses), hepatitis B virus, hepatitis C virus, human coronaviruses 229-E and OC43 and others (causing the common cold, sudden acute respiratory syndrome (SARS), upper respiratory tract infection, human parainfluenza viruses 1 and 3, mumps virus, human parainfluenza viruses 2, 4a and 4b, measles virus, human respiratory syncytial virus, rabies virus, Marburg virus, Ebola virus, influenza A, B and C viruses, Lymphocytic choriomeningitis (LCM) virus, Lassa virus, human immunodeficiency viruses 1 and 2, Vaccinia, Subfamily: human herpes viruses 1 and 2, herpes virus B, Epstein-Barr virus, smallpox virus, Yellow fever virus, cowpox virus, poliovirus, Norwalk virus, molluscum contagiosum virus, and any other lipid-containing virus.

Alternative viruses to be treated with the method include the various immunodeficiency viruses including human (HIV), simian (SIV), feline (FIV), as well as any other form of immunodeficiency virus. Other preferred viruses to be treated with the method include hepatitis in its various forms, especially, hepatitis B and hepatitis C. It is to be understood that the present method is not limited to the viruses provided above. All viruses, including those containing lipid in their viral envelope, are included within the scope of the disclosure.

Viruses within the broad category of non-lipid enveloped are included within the scope of this patent. These viruses include hepatitis A, Parvoviruses (bovine, porcine, canine and human B19), Adenoviruses (Adenovirus 2 and canine hepatitis), Picornaviruses (Poliovirus 1 and 2, Coxsackie A-9 and Echo 11), and Reoviruses (Reovirus 3, Blue Tongue and Colorado tick fever), Astroviruses, Birnaviruses, Calciviruses, Circoviruses, Papillomaviruses, Papoviruses (SV-40), Polyomaviruses, and Rotaviruses.

Maintenance of the Biological Integrity of Treated Biological Fluids

In one embodiment the inactivation method provides a means to inactivate viruses while maintaining the biological integrity of the product for its intended use. Furthermore, the method allows inactivation of viruses at temperatures compatible with maintaining the biological integrity of biological fluids. For blood products, the biological integrity of plasma may be measured by the functionality of its protein components either in whole plasma or after separation into plasma fractions. The biological integrity of red blood cell and platelet preparations may be determined by the methods and criteria known by those skilled in the art and are similar to those used in establishing the suitability of storage and handling protocols. In practical terms, the biological integrity of a biological fluid is a fluid that subsequent to the inactivation method described herein, has sufficiently maintained its replacement value for intended use in vivo or in vitro. Specifically, maintaining the biological integrity of a growth media can be measured as the sufficient retention of the functionality of the media to support growth and its other indicated uses subsequent to the inactivation process described herein.

Mechanism(s) of Inactivation of Viruses with Ozone

Ozone has been postulated to inactivate viruses through a number of potential mechanism(s). Direct contact of lipid-enveloped viruses with ozone may result in the oxidation of polyunsaturated fatty acids thereby increasing membrane fluidity and resulting in viral inactivation. With non-lipid enveloped viruses, inactivation has been considered to occur through a direct oxidational decomposition of the capsid/protein shell of the virus, thus exposing its DNA and RNA components to open attack; the presence of DNA and RNA catabolism products has been demonstrated empirically. Alternatively, ozone may react with either the lipid-envelope of the virus or other components in a virally contaminated preparation to produce secondary products of ozonation, including lipid peroxides that in turn may participate in the viral inactivation. Other mechanism(s) include the perturbation of lipids or proteins in the viral coat disrupting the ability of the virus to bind to cell surface receptors thereby reducing infectivity.

Lipid Peroxidation

A method is provided of inactivating viruses while maintaining the biological integrity of the biological fluid producing a non-infectious biological fluid. The method involves subjecting a quantity of a fluid containing a virus, including lipid-enveloped viruses, to an amount of ozone delivered by an ozone delivery system. Inactivation of viruses in biological fluids generates byproducts of ozonation, including lipid peroxides that are a result of the absorption of an absorbed-dose of ozone from the delivered-ozone by the fluid. The lipid peroxides are quantifiable by a variety of assays.

Oxidation of polyunsaturated fatty acids involves an allylic hydrogen abstraction followed by insertion of molecular oxygen; the resulting peroxyl radicals abstract hydrogens to form lipid peroxides. Although the mechanism(s) by which ozone mediates a lipid peroxidation are not known with certainty, it has been demonstrated that ozone reacts directly with carbon-carbon double bonds in unsaturated fatty acids and that free radical lipid peroxides can be detected by electron spin resonance. These lipid peroxides are relatively unstable compounds and decompose to form a series of reactive carbonyl compounds including malondialdehyde (‘MDA’) and 4-hydroxyalkenals (‘HAE’). Measurement of either MDA or HAE may be utilized as an indicator of lipid peroxidation.

MDA Assay

The assay for the quantification of lipid peroxides can be performed according to the thiobarbituric acid (‘TBA’) procedure where the formation of the malondialdehyde-TBA adduct results in a fluorometrically assayable species at 532 nm under acidic conditions. The test measures the amount of malondialdehyde, a decomposition product of lipid peroxides. The total amount of malondialdehyde MDA measured reflects the amount formed from lipid peroxides during the testing methodology, and the relatively minor component formed naturally, in vivo. During the assay itself, malondialdehyde is formed from lipid peroxides. Thus the procedure assays ‘native’ lipid peroxides as well as ‘native’ malondialdehyde, both of which are degradation products of radical-induced lipid autoxidation. An alternative method to the fluorometric determination in the thiobarbituiric acid assay for lipid peroxide quantification is through the use of High Pressure Liquid Chromatography. The procedure involves forming the thiobarbituric acid-malondialdehyde adduct, separating it on an uBondapak C₁₈ column, and measuring the absorbance at 546 nm.

An alternative method for quantification of lipid peroxides is based upon the formation of a stable chromophore between the MDA degradation product of lipid peroxides and N-methyl-2-phenylindole under acidic conditions, and assayable by spectroscopy at 586 nm. Both fluorometric and HPLC thiobarbituric detection methodologies as well as the spectrophotometric assay utilizing an N-methyl-2-phenylindole-MDA adduct for quantification of lipid peroxides yield equivalent results.

HAE Assay

Analysis of the formation of 4-hydroxyalkenals (‘4-HAE’) can also be utilized as a measure of lipid peroxidation. Although 4-HAB represents relatively minor degradation products of lipid peroxidation as compared to MDA, they can also be used as an indicator of lipid peroxidation. The most abundant 4-hydroxyalkenal formed in lipid peroxidation is 4-hydroxy-2-nonenal (‘HNE’). The assay for HAE is based upon the formation of a stable chromophore with N-methyl-2-phenylindole under acidic conditions. In contrast to the assay for MDA, the acidic conditions for HAE assay involve the use of methanesulfonic acid. HAE may either be assayed individually or in conjunction with MDA by spectroscopy at 586 nm.

Absorption of ozone by a biological fluid will cause, as a byproduct of oxidation, reactive oxygen intermediates including the formation of a variety of lipid peroxides (‘LPs’). These LPs are a product of ozonation at the various sites of unsaturation found in the carbon chain lengths found in the aliphatic portion of the fatty acids that compose alipids. Generally, these LPs can be quantified by conventional methods such as the formation of a malondialdehyde or hydroxyalkenal adduct by the assays described above. Those skilled in the art will appreciate that other assays are available for the quantification of lipid peroxides. These adducts are subsequently quantified by fluorescence or absorption spectroscopy against an established standard curve. However, the high reactivity and concomitant short half-life of those LPs derived from 5 carbon chain lengths or less in an aqueous fluid, and which form more stable aldehydes, ketones, alcohols or other relatively stable oxygen containing compounds that do not contain a peroxide component, are precluded from such measurement. In contrast, a large number of LPs that are derived from fatty carbon chain lengths in excess of 5 carbons are sufficiently stable and therefore measurable. The wide variety of LPs that are measured by these assays are represented as a total aggregate number of LPs.

A direct relationship can be derived between the absorbed-dose of ozone absorbed by a biological fluid and the aggregate number of LPs produced. Quantifiable lipid peroxides are pharmacologically active and possess intrinsic antiviral activity. A quantifiable pharmacologically antiviral amount of lipid peroxides generated when a biological fluid has absorbed an absorbed-dose of ozone is considered a drug-blood product. Products of ozonation, including lipid peroxides can be quenched by the addition of a biocompatible agent. These biocompatible agents may include all known antioxidants including Vitamin A, Vitamin E, other tocopherol-containing compounds, glutathione, ascorbic acid, curcumin and activated charcoal. In the event that activated charcoal is chosen as the agent, subsequent to quenching the products of ozonation, the activated charcoal may be removed. Furthermore, the use of activated charcoal may be used to remove the products of ozonation, including lipid peroxides. Subsequent to the contact of the activated charcoal with the ozone absorbed biological fluid, the charcoal may be removed.

Ozone Delivery System

An ozone delivery system delivers a measured amount of an ozone/oxygen admixture and is able to measure, control, report and differentiate between the delivered-ozone and absorbed-dose of ozone. The system provides a controllable, measurable, accurate and reproducible amount of ozone that is delivered to a controllable, measurable, accurate and reproducible amount of a biological fluid and controls the rate of ozone absorption by the fluid resulting in a quantifiable absorbed-dose of ozone thereby producing a non-infectious biological product. The system may accomplish this by using:

A manufacturing component, control components, measuring components, a reporting component and calculating component (such as an ozone generator, gas flow meter, fluid pump, variable pitch platform, data acquisition device, inlet ozone concentration monitor, and exit ozone concentration monitor) that cooperate to manufacture and deliver a measured, controlled, accurate and reproducible amount of ozone, the delivered-ozone, to a fluid through the use of a gas-fluid contacting device that provides for the interface between the ozone/oxygen admixture and fluid. Using control components, measuring components, a reporting component and calculating component.

Using a gas flow meter, fluid pump, variable pitch platform, data acquisition device, inlet ozone concentration monitor, and exit ozone concentration monitor that cooperate, the system may instantly differentiate the delivered-ozone from the absorbed-dose of ozone.

The system utilizes (a gas flow meter, fluid pump, variable pitch platform, data acquisition device, inlet ozone concentration monitor, and exit ozone concentration monitor) control components, measuring components, a reporting component and calculating component that cooperate and instantly report data that may include the delivered-ozone, residual-ozone, absorbed-dose of ozone, interface-time, elapsed-time, and, the amount and flow rate of the fluid delivered to the gas-contacting device.

1. Construction Materials

The gas-contacting surfaces of the ozone delivery system including gas-fluid contacting devices are constructed from ozone-inert materials to avoid consumption of ozone. Ozone-inert materials include stainless steel, borosilicate, quartz, ceramic composites, PFA (copolymer of tetrafluoroethylene and perfluorinated vinyl ether from the perfluoroalkoxy group) and PTFE (polytetrafluoroethylene, TEFLON), and are further defined as being non-reactive within the concentration range of ozone manufactured and delivered by an ozone delivery system. A delivery system including one or more gas-fluid contact devices may be constructed without the inclusion of any fluoropolymers, polyfluoroethylene, PFA or PTFE materials, in the event PTFE becomes a health concern.

2. Gas Flow

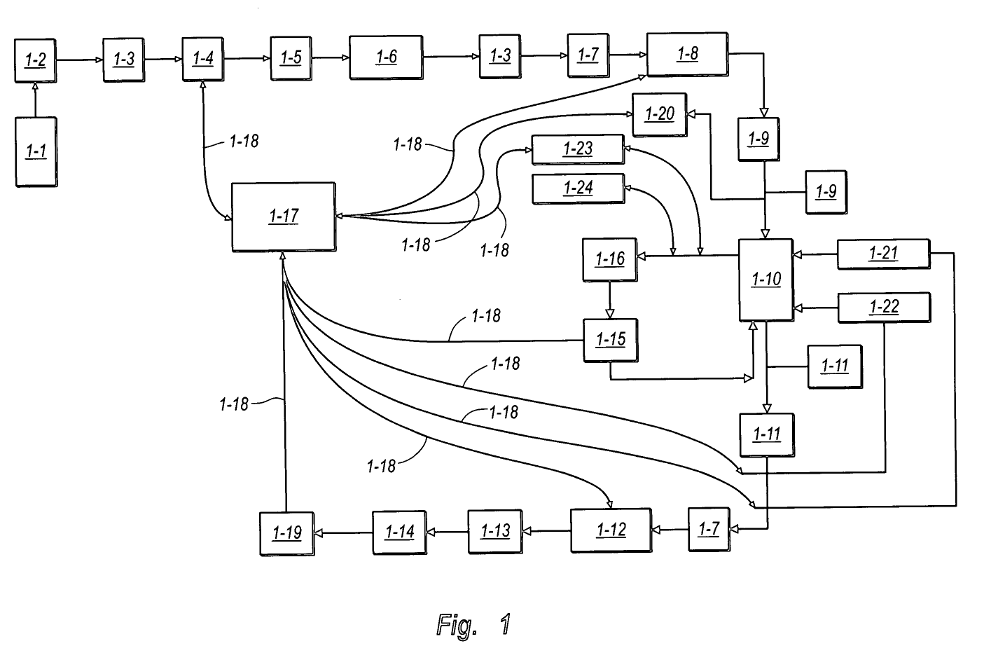

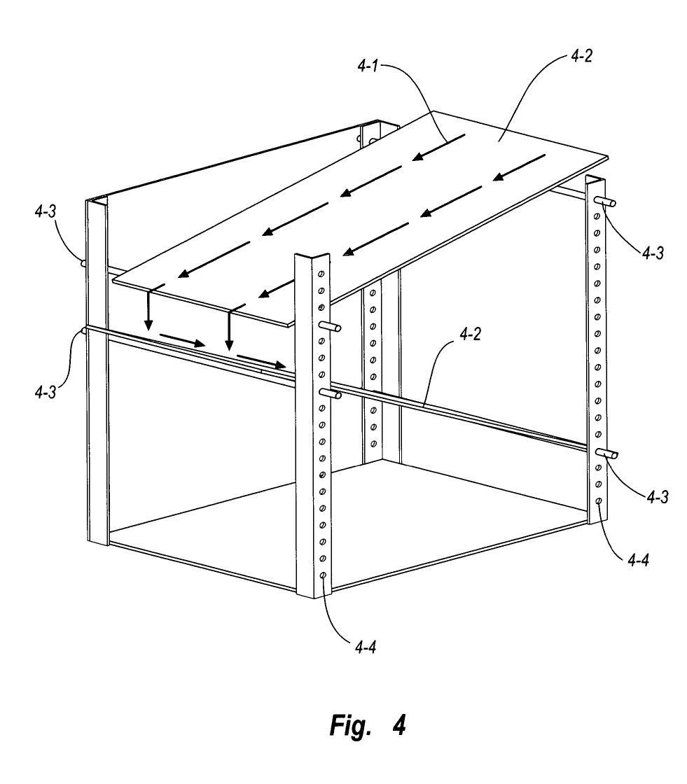

Medical grade oxygen is the source gas utilized, as lesser grades of oxygen may include nitrogenous contaminants resulting in the formation of toxic nitrous oxides. FIG. 1 illustrates that the oxygen flows from a pressurized cylinder (1-1), through a regulator (1-2), through a particle filter (1-3), through a flow meter (1-4) where the oxygen and subsequent ozone/oxygen admixture flow rate is controlled and measured, through a pressure release valve (1-5), through an ozone generator (1-6) where the concentration of the ozone/oxygen admixture is manufactured and controlled and where the admixture volume contains the delivered-ozone. The ozone/oxygen admixture flows through a particle filter (1-3) to remove particulates, and through an optional moisture trap (1-7), to reduce moisture. The admixture proceeds through an ozone inlet concentration monitor (1-8) that measures and reports the inlet ozone concentration of the ozone/oxygen admixture that contains the delivered-ozone. This real-time measurement may be based on ozone's UV absorption characteristics as a detection methodology. The ozone/oxygen admixture then passes through a set of valves (1-9) used to isolate a gas-fluid contacting device for purging of gasses. The ozone/oxygen admixture may pass an optional humidity sensor (1-20) where humidity may be measured and recorded, through a gas-fluid contacting device (1-10) where it interfaces with a fluid. The interface-time between an ozone/oxygen admixture and a fluid may be controlled through adjustment of the variable pitch platform as illustrated in FIG. 4, the fluid pump (1-15), and the time controlling capacity of the data acquisition device (1-17). The interface-time can be measured by the data acquisition device (1-17). Temperature (1-21) and pressure (1-22) may be measured by the use of temperature and pressure sensors, respectively, inserted into their respective ports. The resultant ozone/oxygen admixture containing the residual-ozone then exits the gas-fluid contacting device and flows through the exit purge valves (1-11), through a moisture trap (1-7), through an exit ozone concentration monitor (1-12), which may utilize a similar detection methodology as ozone concentration monitor (1-8), and that measures and reports the exit ozone/oxygen admixture concentration. The exiting ozone/oxygen admixture then proceeds through a gas drier (1-13), through an ozone destructor (1-14) and a flow meter (1-19).

3. Fluid Flow

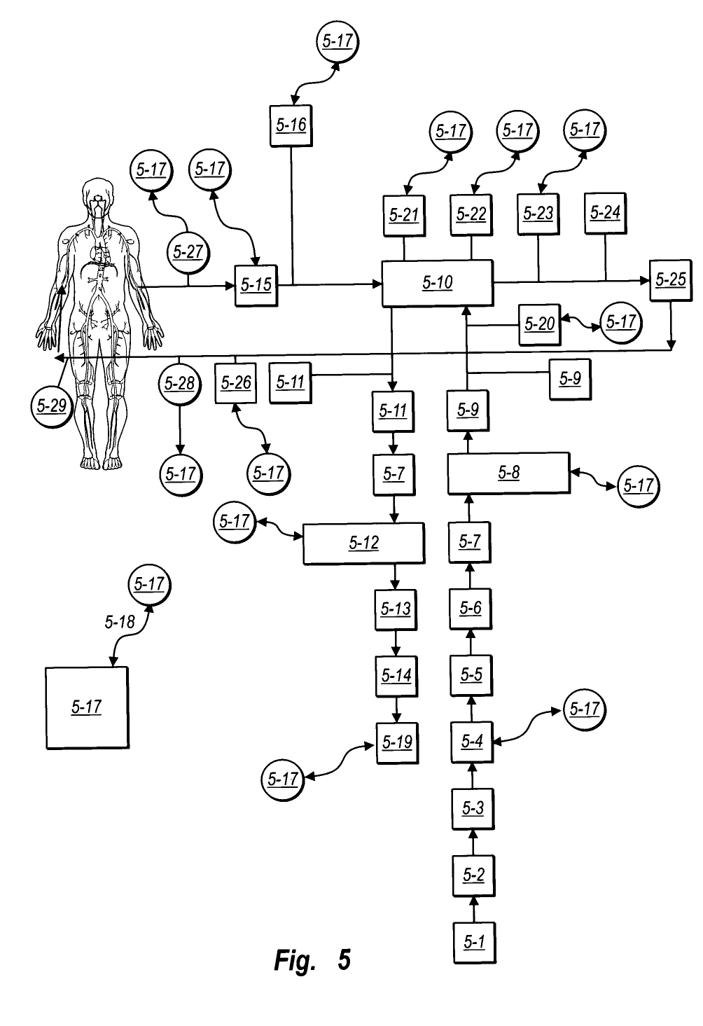

FIG. 1 further illustrates that a fluid flows through tubing, from the fluid pump (1-15), into the gas-fluid contacting device (1-10) where it interfaces with an ozone/oxygen admixture containing the delivered-ozone. Insertion ports for temperature and pressure sensors may be located in the gas-fluid contacting device for the measurement of temperature and pressure, respectively. After interfacing with the ozone/oxygen admixture, the fluid exits into tubing that may contain a port for an optional absorbed oxygen sensor (1-23) followed by a fluid access port allowing for fluid removal (1-24) and into an optional reservoir (1-16), where if configured in a closed loop, the fluid is circulated in a repetitive manner. Other fluid loop configurations may be utilized, including but not limited to, configurations similar to those used in dialysis for mammalian applications, for example as depicted in FIG. 5.

A data acquisition device (1-17), such as DAQSTATION (Yokogawa), for example, has time measurement capabilities, reports, stores and monitors data instantly and in real-time, and performs various calculations and statistical operations on data acquired. All data is transmitted to the data acquisition device through data cables (1-18), including: data from ozone concentration monitors (1-8) and (1-12), flow meters (1-4) and (1-19), humidity sensor (1-20), temperature sensor (1-21), pressure sensor (1-22), fluid pump (1-15), and absorbed oxygen sensor (1-23). The elapsed time, a composite of both the interface time and the period of time that the fluid circulates through the other elements of the apparatus can be measured and controlled through the data acquisition device (1-17).

4. Measurement of Delivered-Ozone, Residual-Ozone and Absorbed-Dose of Ozone

The ozone delivery system utilizes measuring components, reporting components and calculating components (such as an inlet ozone concentration monitor, exit ozone concentration monitor, gas flow meter, fluid pump, data acquisition device) that cooperate together to determine certain calculated-data including the delivered-ozone, the residual-ozone and the absorbed-dose of ozone.

Delivered-ozone is an amount of ozone calculated by multiplying the measured volume of ozone/oxygen admixtures, as reported by gas flow meters, by the measured concentration of ozone within the ozone/oxygen admixture as it enters the gas-fluid contacting device, as reported by the inlet ozone concentration monitor. The measured volume of ozone/oxygen admixtures is calculated by multiplying the measured gas flow reported by gas flow meters, by the elapsed-time, as measured by the time measuring and calculating capability of the data acquisition device.

Residual-ozone is an amount of ozone calculated by multiplying the measured volume of ozone/oxygen admixtures, as reported by gas flow meters, by the measured concentration of ozone within the ozone/oxygen admixture exiting the gas-fluid contacting device, as reported by the exit ozone concentration monitor. The measured volume of ozone/oxygen admixtures is calculated by multiplying the measured gas flow reported by gas flow meters, by the elapsed-time, as measured by the time measuring and calculating capability of the data acquisition device.

The absorbed-dose of ozone is an amount of ozone calculated by subtracting the amount of residual-ozone from the amount of delivered-ozone. The absorbed-dose of ozone may range from 1 to 10,000,000 micrograms per milliliter of fluid, and may be between 1 and 10,000 ug per milliliter of fluid.

All measured-data, including measured data from the gas flow meters, inlet and exit ozone concentration monitors, the fluid pump, temperature sensors, pressure sensors, absorbed oxygen sensor and humidity sensors are transmitted to a data acquisition device. The data acquisition device has time measuring capabilities and instant, real-time reporting, calculating and data storing capabilities to process all measured data. The data acquisition device may use any measured data or any combination of measured data as variables to produce calculated-data. Examples of calculated-data may include delivered-ozone, residual-ozone, absorbed-dose of ozone, absorbed-dose of ozone per unit volume of fluid, and the absorbed-dose of ozone per unit volume of fluid per unit time.

5. Variables and Equipment

An ozone delivery system includes an ozone generator (1-6) for the manufacture and control of a measured amount of an ozone/oxygen admixture and where the admixture volume contains the delivered-ozone. A commercially available ozone generator capable of producing ozone in a concentration range between 10 and 3,000,000 ppmv of ozone in an ozone/oxygen admixture may be employed. Ozone/oxygen admixture concentrations entering the gas-fluid contacting device are instantly and constantly measured in real time, through an ozone concentration monitor (1-8) that may utilize UV absorption as a detection methodology. A flow meter (1-4) controls and measures the delivery of the delivered-ozone in an ozone/oxygen admixture to the gas-fluid contacting device at a specified admixture flow rate. Ozone/oxygen admixture flow rates are typically in the range between 0.1 and 5.0 liters per minute.

Measurement of the humidity of the ozone/oxygen admixture delivered to the gas-fluid contacting device may be included through the use of a humidity sensor. A humidity sensor port (1-20) may be provided in the ozone/oxygen admixture connecting tubing, however, it can be placed in a variety of locations. For example, the humidity sensor may be located in the connecting tubing prior to the admixture's entrance into gas-fluid contacting device.

Measurement of the temperature within the gas-fluid contacting device during the interface-time may be provided by inclusion of a temperature sensor port (1-21) in the gas fluid contacting device through which a temperature sensor may be inserted. The temperature at which ozone/oxygen admixtures interface fluids ranges from 4° to 100° C., and may be performed at ambient temperature, 25° C., for example. The temperature at which the interface occurs can be controlled by placing the gas-fluid contacting device, optional reservoir, and both gas and fluid connecting tubing in a temperature controlled environment, and/or by the addition of heating or cooling elements to the gas-fluid contact device.

Measurement of the pressure within the gas-fluid contacting device during the interface-time is provided by inclusion of a pressure sensor port (1-22) in the gas-fluid contacting device through which a pressure sensor may be inserted. The pressure at which an ozone/oxygen admixtures interfaces with a fluid ranges from ambient pressure to 50 psi and may be performed between ambient pressure and 3 psi, for example. A pressure sensor port may be provided in each gas-fluid contacting device to measure and report the pressure at which the interface occurs.

The concentration of the ozone/oxygen admixtures exiting the gas-fluid contacting device and where the admixture volume contains the residual-ozone, are instantly and constantly measured in real time through an exit ozone concentration monitor that may utilize UV absorption as a detection methodology (1-12).

A fluid pump (1-15) controls and measures the flow rate of the fluid delivered to the gas-fluid-contacting device at a specified fluid flow rate. Fluid flow rates through the gas-fluid contacting device typically will range from 1 ml to 100 liters per minute, and for example, may be between 1 ml to 10 liters per minute. The fluid is generally contained within a closed-loop design and may be circulated through the gas-fluid contacting device once or multiple times.

Measurement of the amount of oxygen absorbed into a fluid while it interfaces with the ozone/oxygen admixture within the gas-fluid contacting device may be provided through the use of an absorbed oxygen sensor. The sensor is inserted within the absorbed oxygen sensor port (1-23) located in the tubing as it exits the gas-fluid contacting device. Measurement of absorbed oxygen may be recorded in various units, including ppm, milligrams/liter or percent saturation.

The system also includes a fluid access port (1-24) for fluid removal. The port is generally located in the tubing member after the fluid exits through the fluid exit port of the gas-fluid contacting device and prior to the optional reservoir (1-16).

A data acquisition device (1-17), such as DAQSTATION (Yokogawa), for example, has time measurement capabilities, reports, stores and monitors data instantly and in real-time, and performs various calculations and statistical operations on data acquired. Data is transmitted to the data acquisition device through data cables (1-18), including: data from ozone concentration monitors (1-8) and (1-12), flow meters (1-4) and (1-19), humidity sensor (1-20), temperature sensor (1-21), pressure sensor (1-22), fluid pump (1-15) and absorbed oxygen sensor (1-23).

One of skill in the art will appreciate that components of an ozone delivery system may be replaced by those of technical equivalence.

Calculated-data may include delivered-ozone, residual-ozone, and the absorbed-dose of ozone. Measurement of the volume of the ozone/oxygen admixture delivered can be calculated though data provided from the flow meter (1-4) and the time measurement capability of the data acquisition device (1-17). Measurement of the volume of fluid delivered to the gas-fluid contacting device (1-10) can be calculated by the data acquisition device (1-17) utilizing fluid flow rate data transmitted from the fluid pump (1-4).

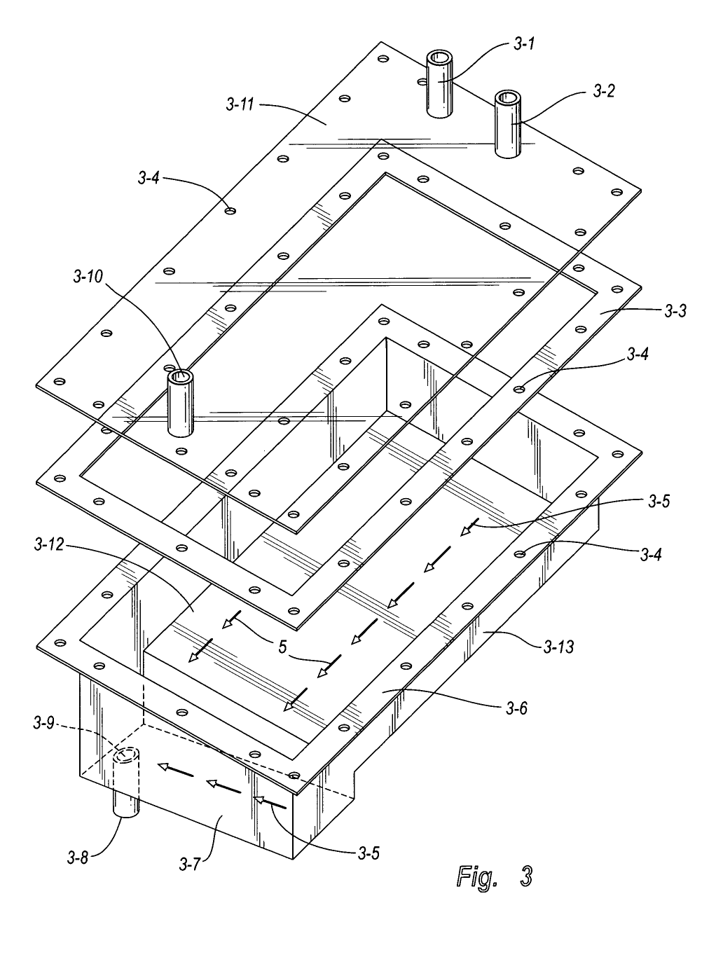

The elapsed-time can be measured and controlled through the data acquisition device (1-17). The elapsed-time that the fluid circulates through the apparatus including the gas-fluid contacting device and is interfaced with an ozone/oxygen admixture can vary, generally for duration of up to 120 hours. The interface-time may also be measured by the time measuring capacity of the data acquisition device (1-17). The interface-time between a fluid and an ozone/oxygen admixture may be controlled through a composite of controls. These controls include: the angle of the gas fluid contacting device (as illustrated in FIGS. 3 and 4), the fluid flow rate via fluid pump, and the time controlling capacity of the data acquisition device. The interface-time may vary in duration of up to 720 minutes, and generally within duration of up to 120 minutes.

Controllable variables for an ozone delivery system may include: delivered amounts and concentrations of ozone in the entrance ozone/oxygen admixtures, fluid flow rates, admixture flow rates, temperature in the gas-fluid contacting device, interface-time between fluid and admixture; and, the elapsed-time that the fluid may circulate through the apparatus and interface with an ozone/oxygen admixture.

Measurable variables may include: ozone/oxygen admixture amounts and flow rates, amounts and concentrations of ozone in the entrance and exit ozone/oxygen admixtures, fluid flow rates, temperature and pressure in the gas-contacting device, humidity of the entrance admixture to the gas-fluid contacting device, absorbed oxygen by the fluid, interface-time and elapsed-time.

Data representing controllable variables and measurable variables acquired by the apparatus allows for a variety of calculations including: delivered-ozone, residual ozone, absorbed-dose of ozone, absorbed-dose of ozone per unit volume of fluid, and the absorbed-dose of ozone per unit volume of fluid per unit time.

Continuous Loop Configuration

FIG. 5 illustrates blood from a patient being extracorporeally interfaced with an ozone/oxygen admixture. Blood may be circulated in a continuous loop format in a venovenous extracorporeal exchange format as will be appreciated by one of skill in the art. As an example, this continuous loop can be established through venous access of the antecubital veins of both right and left arms. Prior to establishing an extracorporeal circuit, a patient may optionally be anticoagulated with heparin or any other suitable anticoagulant known to those skilled in the art.

1. Gas Flow for Continuous Loop

The oxygen flows from a pressurized cylinder (5-1), through a regulator (5-2), through a particle filter (5-3) to remove particulates, through a flow meter (5-4) where the oxygen and subsequent ozone/oxygen admixture flow rate is controlled and measured. The oxygen proceeds through a pressure release valve (5-5), through an ozone generator (5-6) where the concentration of the ozone/oxygen admixture is manufactured and controlled and where the admixture volume includes the delivered-ozone. The ozone/oxygen admixture flows through an optional moisture trap (5-7), to reduce moisture. The admixture proceeds through an inlet ozone concentration monitor (5-8) that measures and reports the inlet ozone concentration of the ozone/oxygen admixture that contains the delivered-ozone. This real-time measurement may be based on ozone's UV absorption characteristics as a detection methodology. The ozone/oxygen admixture then passes through a set of valves (5-9) used to isolate a gas-fluid contacting device for purging of gasses. The ozone/oxygen admixture may pass an optional humidity sensor (5-20) where humidity may be measured and recorded, and into a gas-fluid contacting device (5-10) where it interfaces with fluid. The interface-time between fluid and ozone/oxygen admixture may be controlled through adjustment of the variable pitch platform as illustrated in FIG. 4, fluid pump (5-15) and the time controlling capacity of the data acquisition device (5-17). The interface-time may then be measured by the data acquisition device (5-17). Temperature (5-21) and pressure (5-22) may be measured by the use of optional temperature and pressure sensors, respectively, inserted into their respective ports. The resultant ozone/oxygen admixture containing the residual-ozone exits the gas-fluid contacting device and flows through the exit purge valves (5-11), through a moisture trap (5-7), through an exit ozone concentration monitor (5-12), which may utilize a similar detection methodology as the inlet ozone concentration monitor (5-8), that measures and reports the exit ozone/oxygen admixture concentration. The exiting ozone/oxygen admixture then proceeds through a gas drier (5-13), through an ozone destructor (5-14) and a flow meter (5-19).

2. Fluid Flow for Continuous Loop

Intravenous blood flows from the patient through tubing through a pressure gauge (5-27) which monitors the pressure of the blood flow exiting the patient. Generally, the pressure of the blood exiting the patient ranges from a negative pressure of 100-200 mm Hg, and may be between a negative pressure of 150 and 200 mm Hg, with a maximum cutoff pressure of minus 250 mm Hg. The blood flows through a fluid pump (5-15) and is optionally admixed with heparin or other suitable anticoagulant as provided by an optional heparin pump (5-16). The blood then passes through the gas-fluid contacting device (5-10) where it interfaces with the ozone/oxygen admixture containing the delivered-ozone. Ports for the insertion of sensors may be located in the gas-fluid contacting device for the measurement of temperature and pressure, respectively. After interfacing with the ozone/oxygen admixture, the fluid exits into tubing that may contain a port for an optional absorbed oxygen sensor (5-23) followed by a fluid access port (5-24). The blood continues through an air/emboli trap (5-25) that removes any gaseous bubbles or emboli. The blood then continues through a fluid pump (5-26) and then into a pressure gauge (5-28) which monitors the pressure of the blood flow before returning to the patient. Generally, the pressure of the blood entering the patient ranges from a pressure of 100-200 mm Hg, and may be between 150 and 200 mm Hg, with a maximum cutoff pressure of 250 mm Hg. The blood continues through a priming fluid access port (5-29) that allows for the removal of the priming fluid from the extracorporeal loop. The blood is then re-infused directly into the patient.

A data acquisition device (5-17), such as DAQSTATION (Yokogawa), for example, has time measurement capabilities, reports, stores and monitors data instantly and in real-time, and performs various calculations and statistical operations on data acquired. All data is transmitted to the data acquisition device through data cables (5-18), including: data from ozone concentration monitors (5-8) and (5-12), flow meters (5-4) and (5-19), humidity sensor (5-20), temperature sensor (5-21), pressure sensor (5-22), fluid pumps (5-15) and (5-26), pressure gauges (5-27) and (5-28), and absorbed oxygen sensor (5-23). The elapsed time, a composite of both the interface time and the period of time that the fluid circulates through the other elements of the apparatus can be measured and controlled through the data acquisition device (5-17).

Other possible configurations for an extracorporeal blood circuit known to those skilled in the art are included within the spirit of this disclosure.

Gas-Fluid Contacting Devices

One or more gas-fluid contacting devices may be included in an ozone delivery system to increase the surface area of a fluid to be treated allowing for an increase in the mass transfer efficiency of the ozone/oxygen admixture. Gas-fluid contacting devices may encompass the following properties: closed and isolated from the ambient atmosphere, gas inlet and outlet ports for the entry and exit of ozone/oxygen admixtures, fluid inlet and outlet ports for the entry and exit of a fluid. They may also include components such as a temperature sensor, pressure sensor and data acquisition device for the measurement and reporting of temperature and pressure within a gas-fluid contacting device. These devices may generate a thin film of the fluid as it flows within a gas-fluid contacting device, and may be constructed from ozone-inert construction oz>materials including, quartz, ceramic composite, borosilicate, stainless steel, PFA and PTFE.

Gas-fluid contacting devices include designs that encompass surfaces that may be horizontal or approaching a horizontal orientation. These surfaces may include ridges, indentations, undulations, etched surfaces or any other design that results in a contour change and furthermore, may include any pattern, regular or irregular, that may disrupt the flow, disperse the flow or cause turbulence. These surfaces may or may not contain holes through which a fluid passes through. The surface of the structural elements may have the same or different pitches. Designs of gas-fluid contacting devices may include those that involve one or more of the same shaped surfaces or any combination of different surfaces, assembled in any combination of ways to be encompassed within the device may include cones, rods, tubes, flat and semi-flat surfaces, discs and spheres.

The interface between an ozone/oxygen admixture and a fluid may be accomplished by the use of a gas-fluid contact device that generates a thin film of the fluid that interfaces with the ozone-oxygen admixture as it flows through the device. One of skill in the art will appreciate that generation of any interface that increases the surface area of the fluid and thereby maximizes the contact between a fluid and an admixture, may be used. Additional examples include the generation of an aerosol through atomization or nebulization.

The interface-time within a gas-fluid contacting device is measurable, controllable, calculable and reportable. Furthermore, the interface-time may be for duration of up to 720 minutes, generally however, for duration of up to 120 minutes. Following the interface-time, the fluid exits the gas-fluid contacting device containing the absorbed-dose of ozone. The elapsed-time, a composite of both the interface-time and the time for circulation of a fluid through other elements of an ozone delivery system is also measurable, controllable, calculable and reportable. This elapsed-time is for duration of up to 120 hours.

The pressure at the interface between fluid and ozone/oxygen admixture within a gas-fluid contacting device may be measured. Measurement of pressure within the device may be accomplished through the use of a pressure sensor inserted at the pressure port of the gas-fluid contacting device. The pressure at which an ozone/oxygen admixture interfaces with a fluid ranges from ambient pressure to 50 psi and may be performed between ambient pressure and 3 psi.

The temperature within a gas-fluid contacting device may be controlled by housing the device, such that the device and connecting tubing containing both gas and fluid and an optional reservoir are maintained in a controlled temperature environment. A flow hood that provides for temperature regulation is an example of a controlled temperature environment. Alternatively, the addition of heating or cooling elements to the gas-fluid contact device may provide for the control of temperature. Measurement of temperature within the device may be accomplished through the use of a temperature sensor inserted at the temperature port of a gas-fluid contacting device. The temperature at which ozone/oxygen admixtures interface fluids ranges from 4° to 100° C., and may be performed at ambient temperature, 25° C., for example.

A gas-fluid contacting device may be placed onto any type of agitator platform. The agitator platform may be employed to increase the effectiveness of the O-z ozone/oxygen admixture interface with a fluid being passed through the device.

Gas-fluid contacting devices may be utilized individually or in conjunction with other such devices, whether they are similar or dissimilar in construction, design or orientation. In the event that multiple devices are utilized, either of the same design, or a combination of different gas-fluid contacting devices of different designs, these devices may be arranged one after the other in succession (in series), making a single device out of multiple individual contact devices.

In a series configuration of devices, a fluid flowing through the different contact devices flows in series, from the fluid exit port of one contact device to the fluid entrance port of the next, until passing through all the devices. The ozone/oxygen admixture may flow in a number of arrangements. In one example, the ozone/oxygen admixture flows through different contact devices in series, from the admixture exit port of one contact device to the admixture entrance port of the next. As an alternative example, the ozone/oxygen admixture may flow directly from the admixture source to the entrance port of each different contact device. Another alternative is a combination of the foregoing examples where the ozone/oxygen admixture flows from the exit port of some devices to the entrance port of other devices and in addition, to the entrance of some devices directly from the admixture source. In the event that multiple devices are utilized, the resultant fluid from the terminal device can either be collected or returned to the original device and recirculated.

1. Sphere-Containing Gas-Fluid Contacting Device

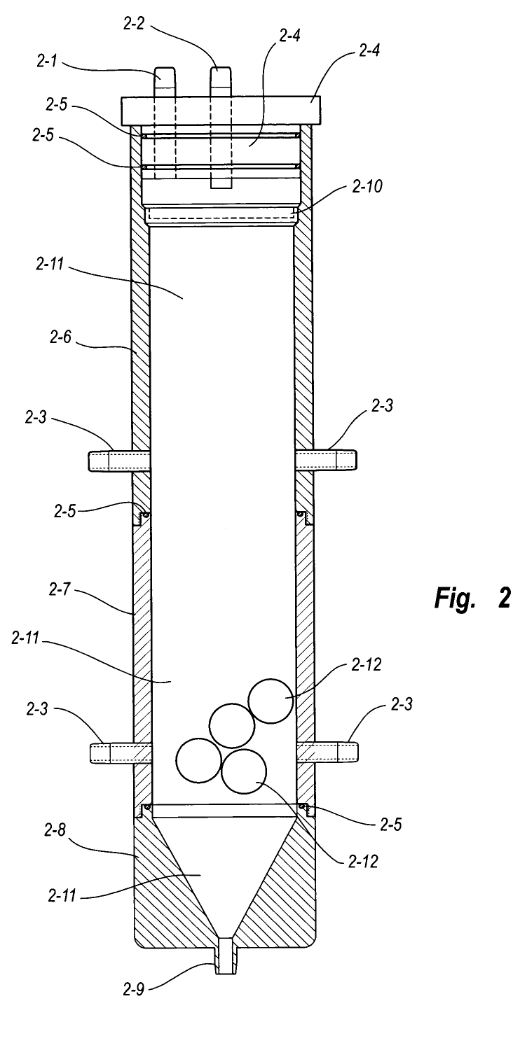

A gas-fluid contacting device, as illustrated in FIG. 2, may consist of an upper housing (2-6), a middle housing of variable thickness (2-7), and a lowerhousing (2-8) of various dimensions with regard to height and internal diameter. One of skill in the art will appreciate that a disparity in dimension is provided for the applicability of a gas-fluid contacting device to fluids of varying volume and viscosity. The device housing includes at least one inlet (2-3) and one exit port (2-1) for ozone/oxygen admixture entrance and exit, respectively. A fluid entrance port (2-2) is positioned at the top of the device permitting entrance and fluid flow is directed in a downward fashion. The top of the device is constructed with a removable cap (2-4) sealed with ozone-inert O-rings (2-5). The ozone/oxygen admixture may flow in a direction similar, counter or in combination thereof to the direction of the fluid flow. A fluid exit port (2-9) may be positioned at the base of the device. Temperature and pressure ports may be included for insertion of temperature and pressure sensors, respectively.

A gas-fluid contacting device may be filled with a number of spheres (2-12), generally of quartz, ceramic composite, borosilicate, PFA or PTFE construction. These spheres may generally range in diameter from 1 to 100 mm, although one of skill in the art will recognize that alternative diameters are possible depending on need. The sphere content and configuration within the cartridge may include; homogenous spherical diameter, heterogeneous spherical diameter, continuous gradient of increasing spherical diameter, continuous gradient of decreasing spherical diameter and discontinuous sets of spheres wherein each set of spheres is of homogenous size but sphere size disparity may exist between the plates.

Regardless of spherical diameter and distribution configuration, the spheres generally occupy approximately seventy-five percent of the internal volume of the device (2-11), however, one of skill in the art will appreciate that alternative volumes are possible. Generally, the total interface surface area of the sphere-containing gas-fluid contacting device can range from 0.01 m² upwards depending on the size of the device, and the sphere diameter chosen. A disk/tray (2-10) may be located above the spheres and is perforated to permit a more homogenous distribution of the fluid across the spherical surfaces upon entrance into the device.

The fluid enters the device from the fluid entrance port (2-2) and flows over the surface of these spheres forming a thin film over the surface of each, and causing turbulence as the fluid flows down through the device. Increasing the surface area of the fluid, by generating a thin film, for example, permits for the maximization of mass transfer of the ozone/oxygen admixture that continuously passes over each sphere. The fluid exits the device through the fluid exit port (2-9).

2. Cylindrical Rod Containing Device

A cylindrical rod-containing device contains a number of cylindrical rods, either solid or hollow in design, whose construction may include quartz, ceramic composite or borosilicate. These rods may generally range in diameter between 3-25 mm but may vary significantly in applications with larger volumes of fluid or different viscosities. In addition, these rods may be constructed with ridges, undulations, indentations or etched surfaces along their length, respectively. The cylindrical rods are secured in place within a housing to maintain a relative equidistance from adjoining rods and the internal walls of the housing.

A fluid entrance port is positioned at the top of the device permitting entrance of a fluid such that flow is gravity directed. Atop the cylindrical rods is a disk that is perforated to permit a more homogenous distribution of the fluid as it enters the device and along the surfaces of both the cylindrical rods and the internal walls of the device housing.

The number of rods contained within a device may vary based upon the interface surface area desired. The total interface surface area of this example gas-fluid contacting device approximates 1.0 m²/meter length of the device or greater depending on the size of the device and the number of rods chosen. Furthermore, one of skill in the art will appreciate that the interface surface area can be substantially increased by incorporating hollow cylindrical (tubes) rods thereby creating a surface area approximating 1.5 m²/meter length of the device.

3. Variable Pitch Device

A gas-fluid contacting device, as illustrated in FIG. 3, may include an enclosed chamber, generally rectangular in shape, whose dimensions may vary based on the interface surface area desired. A fluid enters the device and flows over the bottom surface (3-12) of the chamber to form a thin film. The bottom surface (3-12) may include flat, undulating or ridged designs, may be etched, and may have regular or irregular patterns of any shape or form that disrupt and/or disperse the fluid flow. At the fluid entrance of the device (3-2), construction allows the fluid to distribute evenly along the leading edge of the bottom surface. In contrast, the fluid exit end is constructed with a fluid collection trough (3-7) that is graded toward the drain (3-9) to permit collection of the fluid for exiting. Fluid entrance (3-2) and exit ports (3-8) are positioned at opposite ends of the device.

Fluid flow may be gravity directed (3-5). The top cover (3-11) of the device is secured to the bottom of the device by a flange (3-6) on the base, and uses an ozone-inert gasket (3-3) between the top (3-11) and bottom (3-13) and is attached to the bottom through the use of fasteners that pass through the holes (3-4) in the top cover (3-11), the gasket (3-3), and bottom flange (3-6). One skilled in the art will recognize different sealing and attachment technologies may be employed to attach and seal the top to the bottom, and the method described serves only as an example that should not be considered limiting in scope. The ozone/oxygen admixture may enter the device through the gas inlet port (3-10), and exit the device through the gas exit port (3-1), although these may be reversed depending on the desired direction of the gas flow. The gas may flow in a direction similar, counter or in combination thereof to the direction of the fluid flow. The gas-fluid interface-time is controllable by varying a number of parameters, including adjusting the pitch on a variable pitch platform (See FIG. 4). The pitch is adjustable ranging from 0° (horizontal) to 90° (vertical).

The device exhibited in FIG. 3 can assume a variety of pitches through the use of the platform detailed in FIG. 4. A single or multitude of variable pitch devices (4-2) can be assembled in series on this platform (4-4), that also provides for adjustment and individual pitch variation for each device. The platform has a variety of positions for support rods (4-3) to be inserted on which each device is supported that allows for the variety of pitch desired and resultant fluid flow (4-1).

When arranged in series with other contact devices, interface time between the fluid and ozone/oxygen admixture is controllable, and can be adjusted based on the individual pitch chosen for each device in series, or by adding additional devices to the series. The overall interface surface area will range from 0.01 m² for an individual device, and upwards based on the number of devices serially utilized.

### EXAMPLE 1

An example of data measured and calculated by the ozone delivery system that utilizes a fluid target described herein is included in Table 2. Newborn Calf Serum commercially obtained was utilized as the target fluid. The variable pitch device (FIG. 3) with variable pitch platform (FIG. 4) was employed as the gas-fluid contacting device. The following initial conditions were utilized; 300 ppmv ozone inlet concentration, 145 ml initial fluid volume, 1000 ml per minute gaseous flow rate, 189 ml per minute fluid flow rate counter current to the ozone/oxygen admixture flow. Incremental reductions in fluid volume are due to sampling of fluid through the fluid access port (24).

TABLE 2
NEWBORN CALF SERUM
MEASURED VARIABLES

| | | | | Average Inlet Ozone | Average Exit Ozone |
|---|---|---|---|---|---|
| Elapsed-time | Fluid Volume | Gas Flow Rate | Fluid Flow Rate | Concentration | Concentration |
| (5 min intervals) | (milliliters) | (liters/minute) | (liters/minute) | (ppmv) | (ppmv) |
| 5 | 145 | 0.998 | 0.189 | 305.2 | 38.2 |
| 10 | 143 | 0.972 | 0.189 | 361.5 | 40.4 |
| 15 | 141 | 1.000 | 0.189 | 312.7 | 20.6 |
| 20 | 139 | 1.000 | 0.189 | 314.0 | 37.3 |

CALCULATED VARIABLES

| | Average | | | | |
|---|---|---|---|---|---|
| | Differential Ozone | | | Ozone Absorbed | Absorbed-dose |
| Elapsed-time | Concentration | Delivered-ozone | Residual-ozone | per Interval | of Ozone |
| (minutes) | (ppmv) | (ug) | (ug) | (ug) | (ug) |
| 5 | 267.0 | 3.26E+03 | 4.08E+02 | 2.86E+03 | 2.86E+03 |
| 10 | 321.1 | 7.02E+03 | 8.28E+02 | 3.34E+03 | 6.20E+03 |
| 15 | 292.1 | 1.04E+04 | 1.06E+03 | 3.12E+03 | 9.32E+03 |
| 20 | 276.7 | 1.37E+04 | 1.46E+03 | 2.96E+03 | 1.23E+04 |

### EXAMPLE 2

An additional example of data measured and calculated by the system described herein is in Table 3 below. Newborn Calf Serum commercially obtained was utilized as the target fluid. The variable pitch device (FIG. 3) with variable pitch platform (FIG. 4) was employed as the gas-fluid contacting device. The following initial conditions were utilized; 600 ppmv ozone inlet concentration, 137 ml initial fluid volume, 1000 ml per minute gaseous flow rate, 189 ml per minute fluid flow rate counter current to the ozone/oxygen admixture flow. Incremental reductions in fluid volume are due to sampling of fluid through the fluid access port (24).

TABLE 3
NEWBORN CALF SERUM
MEASURED VARIABLES

| Elapsed-time | | | | Average Inlet Ozone | Average Exit Ozone |
|---|---|---|---|---|---|
| (5 minute | Fluid Volume | Gas Flow Rate | Fluid Flow Rate | Concentration | Concentration |
| intervals) | (milliliters) | (liters/minute) | (liters/minute) | (ppmv) | (ppmv) |
| 5 | 137 | 1.000 | 0.189 | 604.2 | 72.0 |
| 5 | 135 | 1.000 | 0.189 | 609.6 | 63.5 |
| 5 | 133 | 1.000 | 0.189 | 606.6 | 70.8 |
| 5 | 131 | 1.000 | 0.189 | 605.3 | 71.7 |

CALCULATED VARIABLES

| | Average | | | | |
|---|---|---|---|---|---|
| | Differential Ozone | | | Ozone Absorbed | Absorbed-dose |
| Elapsed-time | Concentration | Delivered-ozone | Residual-ozone | per Interval | of ozone |
| (minutes) | (ppmv) | (ug) | (ug) | (ug) | (ug) |
| 5 | 532.2 | 6.47E+03 | 7.70E+02 | 5.69E+03 | 5.69E+03 |
| 10 | 546.1 | 1.30E+04 | 1.45E+03 | 5.84E+03 | 1.15E+04 |
| 15 | 535.8 | 1.95E+04 | 2.21E+03 | 5.73E+03 | 1.73E+04 |
| 20 | 533.6 | 2.60E+04 | 2.98E+03 | 5.71E+03 | 2.30E+04 |

### EXAMPLE 3

Another example of data measured and calculated by the system described herein is in Table 4 below. Newborn Calf Serum commercially obtained was utilized as the target fluid. The variable pitch device (FIG. 3) with variable pitch platform (FIG. 4) was employed as the gas-fluid contacting device. The following initial conditions were utilized; 900 ppmv ozone inlet concentration, 145 ml initial fluid volume, 1000 ml per minute gaseous flow rate, 189 ml per minute fluid flow rate counter current to the ozone/oxygen admixture flow. Incremental reductions in fluid volume are due to sampling of fluid through the fluid access port (24).

TABLE 4
NEWBORN CALF SERUM
MEASURED VARIABLES

| Elapsed-time | | | | Average Inlet Ozone | Average Exit Ozone |
|---|---|---|---|---|---|
| (5 minute | Fluid Volume | Gas Flow Rate | Fluid Flow Rate | Concentration | Concentration |
| intervals) | (milliliters) | (liters/minute) | (liters/minute) | (ppmv) | (ppmv) |
| 5 | 145 | 1.000 | 0.189 | 908.1 | 68.0 |
| 5 | 143 | 1.000 | 0.189 | 911.4 | 50.1 |
| 5 | 141 | 1.000 | 0.189 | 904.4 | 46.6 |
| 5 | 139 | 1.000 | 0.189 | 904.7 | 50.9 |

CALCULATED VARIABLES

| | Average | | | | |
|---|---|---|---|---|---|
| | Differential Ozone | | | Ozone Absorbed | Absorbed-dose |
| Elapsed-time | Concentration | Delivered-ozone | Residual-ozone | per Interval | of ozone |
| (minutes) | (ppmv) | (ug) | (ug) | (ug) | (ug) |
| 5 | 840.1 | 9.72E+03 | 7.28E+02 | 8.99E+03 | 8.99E+03 |
| 10 | 861.3 | 1.95E+04 | 1.26E+03 | 9.22E+03 | 1.82E+04 |
| 15 | 857.8 | 2.92E+04 | 1.76E+03 | 9.18E+03 | 2.742+04 |
| 20 | 853.8 | 3.88E+04 | 2.31E+03 | 9.13E+03 | 3.65E+04 |

Pretreatment of the Gas-Fluid Contacting Device

Fluid-contacting surfaces including gas-fluid contacting devices constructed from ozone-inert material(s) may be treated with a human serum albumin (HSA) solution to prevent platelet adhesion, aggregation and other related platelet phenomena in the instances when a biological fluid to be treated contains platelets (i.e. whole blood, platelet concentrates). Generally, HSA solutions ranging between 1 and 10% may be employed. An HSA solution prepared in a biocompatible bacteriostatic buffer solution will be passaged throughout the gas-fluid contacting device. Subsequent to passage, the HSA solution will be drained from the device. The gas-fluid contacting device and all surfaces that are in contact with the biological fluid during the method described are now primed for use with platelet-containing biological fluids.

Viral Inactivation in Fluids

In one embodiment, the biological fluid is whole blood or a derivative thereof. The derivatives include plasma, red blood cell preparations and platelet concentrates. Typically, the present method described would apply to red blood cell and platelet preparations on a single donor basis. That is, an individual blood donation, once fractionated into its respective blood derivative(s), would subsequently be treated to inactivate a virus, including lipid-enveloped viruses, contained therein. The treated blood derivative would then be suitable for in vivo use.

In an alternative embodiment the biological fluid is a unit of whole blood containing one or more viruses, including lipid-enveloped viruses. The whole blood may be fractionated, after being inactivated, into both cellular (red blood cells and platelets) and non-cellular components (plasma) for intended in vivo use.

Conversely, plasma derivatives, commonly pooled from multiple donors, may be the biological fluid to be inactivated as an aggregate product, to be later separated for individual use in vivo. In these instances, large pooled volumes of plasma or plasma-derived factor concentrates would be subjected to inactivation by the method described by incorporating a gas-fluid contacting device compatible with these volumes. Commonly, these applications are performed by the plasma fractionating industry wherein inactivation of viruses is an integral step in the production of plasma fractionates for in vivo use.

### EXAMPLE 4

In one example, fetal calf serum is added to another viral-free biological fluid. Mammalian serum derivatives are a common constituent in culture media and other biological fluids used in the culture of cell-lines, viruses, bacteria and other microorganisms. A common occurrence during the culture of these microorganisms is the simultaneous culture of a viral contaminant in the culture system that renders the culture system useless. It is commonly recognized that the source of this contamination is derived from the addition of the biological fluid component into the culture system. Treatment allows the inactivation of a virus within the biological fluid constituent while maintaining the biological integrity of said biological fluid constituent. The resultant addition of the viral-inactivated biological constituent will therefore not cause viral contamination in the culture system. This example is also applicable to the addition of a biological fluid to a synthetic media wherein the only biological fluid in the system is being treated by the method prior to addition to the synthetic media. Another example of this application is the addition of a biological fluid to a synthetic media in the development of a vaccine.

As previously noted, the inactivation of viruses in biological fluids by absorption of ozone generates byproducts of ozonation, including lipid peroxides. As an example, delivering ozone to a 10% bovine serum fluid in phosphate-buffered saline (“PBS”) at a flow rate of 0.25 liters per minute, consisting of a 1000 ppmv inlet ozone/oxygen admixture concentration at a 2.0 liter per minute admixture flow rate, and for a duration of 30 minutes, results in an absorbed-dose of ozone of approximately 500 ug/ml of fluid. This yields approximately 12 mmole MDA/ml of serum.

In addition, the lipid peroxides generated by this method, measurable by assay, form a dose-response relationship with the absorbed-dose of ozone absorbed by the biological fluid. More specifically, there is a direct correlation between absorbed-ozone and the lipid peroxides generated. Table 5 below depicts the direct relationship between ozone absorbed by a 10% bovine serum in PBS and the degree of lipid peroxide generation as represented by MDA concentration.

[image]

The resultant quantifiable lipid peroxides are considered pharmacologically active and possessing intrinsic antiviral activity, and therefore are considered a drug-blood product.

An aqueous buffered carrier media may be used to absorb ozone directly. Absorption of an absorbed-dose of ozone will cause the generation of byproducts of ozonation in the aqueous buffered carrier media. These byproducts, including lipid peroxides, can be used to inactivate viruses in biological fluids by first generating the byproducts in the carrier media and subsequently adding this byproduct containing-media to the biological fluid containing a virus. Concurrently, the biological integrity of the biological fluid will be maintained. In certain instances however, the biological integrity of the biological fluid may be of no consequence. Byproducts of ozonation in biological fluids, reactive oxygen intermediates including lipid peroxides, may be generated by allowing these byproducts to continue to be generated for a period of time up to 72 hours after the completion of the absorption of ozone and prior to use of the biological fluid. In addition, byproducts of ozonation in aqueous carrier media may be generated by allowing these byproducts to be generated for a period of up to 72 hours after the completion of the absorption of an absorbed-dose of ozone and prior to the introduction of the carrier media into a virus containing biological fluid.

The products of ozonation, reactive oxygen intermediates, including lipid peroxides can be quenched by the addition of a biocompatible agent. These biocompatible agents may include all known antioxidants including Vitamin A, Vitamin E, other tocopherol-containing compounds, glutathione, ascorbic acid, curcumin and activated charcoal. In the event that activated charcoal is chosen as the agent, subsequent to quenching the products of ozonation, the activated charcoal may be removed. Furthermore, the method further instructs that the use of activated charcoal may remove the products of ozonation, reactive oxygen intermediates including lipid peroxides. Subsequent to the contact of the activated charcoal with the ozone absorbed biological fluid, the charcoal may be removed.

### EXAMPLE 5

Examples of the inactivation of model viruses are included in Table 6 below. These viruses represent a number of families, differ in the genomic composition, include both lipid-enveloped and non-enveloped virions, and vary in their respective symmetries. Virally contaminated fluids (a number of different concentrations of bovine serum products spiked with the indicated virus) varied in serum or protein concentration as indicated and were constituted in phosphate buffered saline. Inactivation was determined by either plaque forming or focal forming unit assays in target cell lines, as appropriate. Elapsed-time indicates the period of time required to affect the indicated level of inactivation.

Parallel cytotoxicity, cell viability and cell proliferation studies were performed (data not shown) without any virus in these various serum preparations. In each instance, the serum product subjected to the method under identical conditions for viral inactivation yielded a product that maintained sufficient biological integrity to sustain growth and cell proliferation as qualified by analyzing a number of target cell lines.

TABLE 6
Virus Inactivation

| | | | | | Delivered- | Absorbed- | | | Elapsed- |
|---|---|---|---|---|---|---|---|---|---|
| Virus name | | | | Lipid | ozone | dose | | Log | time |
| Strain | Family | Genome | Symmetry | Envelope | (ug) | (ug) | Fluid* | inactivation | (min) |
| Herpes | Herpesviridae | DNA | icosahedral | Yes | 1.20E+05 | 4.65E+04 | 10% CCS | 4.8 | 25 |
| simplex-1 | | | | | | | | | |
| McIntyre | | | | | | | | | |
| Bovine | Parvoviridae | DNA | icosahedral | No | 5.01E+05 | 1.65E+05 | 2% | 3.5 | 120 |
| parvovirus | | | | | | | Fetal Clone | | |
| 3294 | | | | | | | III | | |
| Adenovirus | Adenoviridae | DNA | icosahedral | No | 3.08E+05 | 8.16E+04 | 10% CCS | 5.0 | 60 |
| Type 2 | | | | | | | | | |
| Influenza A | Orthomyoxoviridae | RNA | helical | Yes | 1.54E+05 | 3.47E+04 | 0.125% | 5.5 | 30 |
| Y3301 | | | | | | | BSA | | |
| Vaccinia | Poxviridae | DNA | complex | Yes | 2.31E+05 | 7.46E+04 | 10% CCS | 4.7 | 45 |
| Elstree | | | | | | | | | |
| Vesicular | Rhabdoviridae | RNA | helical | Yes | 3.10E+05 | 2.29E+05 | 100% CCS | 5.1 | 60 |
| stomatitis | | | | | | | | | |
| Indiana | | | | | | | | | |

Non-Fluid Target

In an alternative embodiment, the ozone delivery system is used to inactivate viruses in non-fluid targets. In this embodiment, a gas-fluid contacting device is not employed but rather a contact device applicable to a non-fluid target surface. The ozone delivery system is able to measure, control and report the amount of delivered-ozone, and measure, control and report the absorbed-dose of ozone. For medicinal applications, this embodiment is capable of measuring, reporting and differentiating the amount of delivered-ozone to the target from the absorbed-dose of ozone that is actually utilized by the target.

Examples of non-fluid targets for the delivered-ozone include: external limbs, and, any external tissue surface, including hard, soft and mucosal tissue targets of animals including humans. Internal tissues exposed through a variety of ways, including surgery and trauma may be interfaced with the delivered-ozone. Other targets for the delivered-ozone include: medical implements and instruments, foodstuffs, food handling and storage equipment, pharmaceutical and biological handling and storage equipment, air exchange and conditioning surfaces, microchips and other semi-conductor industry devices.

In this embodiment, an ozone delivery system includes manufacturing, control, measuring, reporting and calculating components (such as an ozone generator, gas flow meter, data acquisition device, inlet ozone concentration monitor, exit ozone concentration monitor) that cooperate together with a contact device for interfacing the delivered-ozone with the target. All gas-contacting surfaces in the system are constructed from ozone-inert construction materials. The structure of the contact device includes: dimensions sufficient to enclose the target, construction design allowing for the enclosed target to be sealed from the external environment, an inlet port for the entrance of the delivered-ozone in an ozone/oxygen admixture, and an exit port for the exit of residual-ozone. In addition, ports for the insertion of sensors to monitor pressure and temperature within the contact device while the delivered-ozone interfaces with the target are optionally provided.

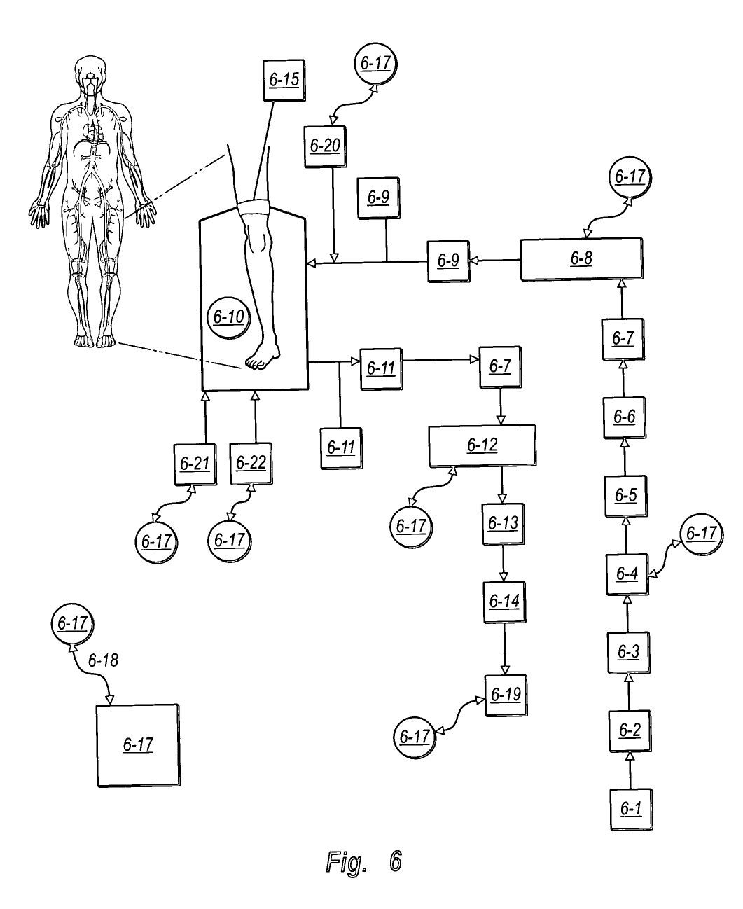

In one example a limb of a patient represents an irregular target surface for delivery of the delivered-ozone in a measured amount of an ozone/oxygen admixture, as illustrated in FIG. 6. The oxygen flows from a pressurized cylinder (6-1), through a regulator (6-2), through a particle filter (6-3) to remove particulates, through a flow meter (6-4) where the oxygen and subsequent ozone/oxygen admixture flow rate is controlled and measured. The oxygen proceeds through a pressure release valve (6-5), through an ozone generator (6-6) where the concentration of the ozone/oxygen admixture is manufactured and controlled and where the admixture volume comprises the delivered-ozone. The ozone/oxygen admixture flows through an optional moisture trap (6-7), to reduce moisture. The admixture proceeds through an inlet ozone concentration monitor (6-8) that measures and reports the inlet ozone concentration of the ozone/oxygen admixture volume that contains the delivered-ozone. This real-time measurement may be based on ozone's UV absorption characteristics as a detection methodology.

The ozone/oxygen admixture then passes through a set of valves (6-9) used to isolate a contact device for purging of gasses. The ozone/oxygen admixture may pass an optional humidity sensor (6-20) where humidity may be measured and recorded, and into a contact-device (6-10) where it is interfaced with the irregular target surface. In one example, the contact device is a “bag-like” structure with the capability to accommodate the irregular contour of the limb. Examples of alternative contacting devices may include chambers of various dimensions, and flexible wrappings or coverings that encompass and contour to the irregularity of a target and includes a closure mechanism for sealing the target from the external environment.

A closure mechanism (6-15) at the site where the limb enters the contact device is provided sealing the enclosed portion of the limb from the environment. The variety of closure mechanisms included within the scope of the disclosure are known to those skilled in the art. The interface-time between the target surface and ozone/oxygen admixture is controlled and measured during the period that the target surface resides in the contact device through control of the time controlling capacity of the data acquisition device (6-17). Temperature (6-21) and pressure (6-22) may be measured by the use of optional sensors inserted into their respective ports. The resultant ozone/oxygen admixture containing the residual-ozone then exits the contact device and flows through the exit purge valves (6-11), through a moisture trap (6-7), through an exit ozone concentration monitor (6-12), which may utilize a similar detection methodology as ozone concentration monitor (6-8), and that measures and reports the exit ozone/oxygen admixture concentration containing the residual-ozone. The exiting ozone/oxygen admixture then proceeds through a gas drier (6-13), through an ozone destructor (6-14) and a flow meter (6-19).

A data acquisition device (6-17), such as DAQSTATION (Yokogawa), for example, reports, stores and monitors data instantly and in real-time, and performs various calculations and statistical operations on data acquired. Data is transmitted to the data acquisition device through data cables (6-18), including: data from ozone concentration monitors (6-8) and (6-12), flow meters (6-4) and (6-19), humidity sensor (6-20), temperature sensor (6-21), and pressure sensor (6-22). The “elapsed-time” for this application is equivalent to the interface time.

According to this non-fluid embodiment, the ozone delivery system delivers an ozone/oxygen admixture containing a measured, controlled and reported amount of delivered-ozone, which enters the inlet port of the contact device encompassing the target. The target interfaces with the delivered-ozone over an elapsed-time where the absorbed-dose of ozone, the amount of ozone absorbed and utilized by the target, is measured, controlled and reported. The residual-ozone is contained within the ozone/oxygen admixture that exits through the exit port of the device. Variations in this embodiment include the measurement of temperature and pressure during the elapsed time for the interface between the target and delivered-ozone.

A method to inactivate viruses in biological fluids while maintaining the biological integrity of the biological fluid to produce a non-infectious biological fluid is disclosed. The method includes subjecting an amount of a fluid containing a virus including lipid-enveloped viruses, to an amount of ozone delivered by an ozone delivery system. The method utilizes an ozone-delivery system for delivering and manufacturing a measured amount of an ozone/oxygen admixture, which is able to measure, control and report and differentiate between delivered-ozone and the absorbed-dose of ozone. The system may include improved gas-fluid contacting devices that maximize gas-fluid mass transfer. All gas contact surfaces of the system, including one or more gas-fluid contact devices are made from ozone-inert construction materials that generally do not absorb ozone or introduce contaminants or deleterious byproducts of oxidation into a fluid.

The method applied to biological fluids, including virally-contaminated fluids, generates byproducts of ozonation, including lipid peroxides that are a result of the absorption of an absorbed-dose of ozone from the delivered-ozone by the fluid. The lipid peroxides are quantifiable by assay for lipid peroxides. A direct relationship between the absorbed-dose of ozone absorbed by a biological fluid and these quantifiable lipid peroxides exists. The lipid peroxides generated are pharmacologically active and possess intrinsic antiviral activity. A quantifiable pharmacologically antiviral amount of lipid peroxides generated when a biological fluid has absorbed an absorbed-dose of ozone is considered a drug-blood product. Furthermore, these lipid peroxides can be quenched with a variety of antioxidants.

The present method may be embodied in other specific forms without departing from its spirit or essential characteristics. The described embodiments are to be considered in all respects only as illustrative and not restrictive. The scope of the system and method of use is, therefore, indicated by the appended claims rather than by the foregoing description. All changes which come within the meaning and range of equivalency of the claims are to be embraced within their scope.

1. A method of inactivating viruses, comprising the steps of:
a) manufacturing an ozone/oxygen admixture to be delivered to a gas-fluid contacting device such that an inlet ozone concentration of the ozone/oxygen admixture is controlled;
b) measuring the inlet ozone concentration of the ozone/oxygen admixture;
c) controlling and measuring the flow rate and the amount of the ozone/oxygen admixture to be delivered to the gas-fluid contacting device;
d) delivering a measured amount of the ozone/oxygen admixture to the gas-fluid contacting device;
e) selecting a biological fluid containing a virus to be delivered to the gas-fluid contacting device;
f) controlling and measuring the flow rate of the biological fluid to be delivered to the gas-fluid contacting device;
g) delivering a measured amount of the biological fluid to the gas-fluid contacting device;
h) interfacing an ozone/oxygen admixture at an inlet ozone concentration with the biological fluid within the gas-fluid contacting device to inactivate the virus, rendering the biological fluid non-infectious, while maintaining the biological integrity of the biological fluid;
i) controlling the time during which the biological fluid interfaces with the inlet ozone concentration of the ozone/oxygen admixture;
j) releasing an ozone/oxygen admixture at an exit concentration from the gas-fluid contacting device after the ozone/oxygen admixture has interfaced with the biological fluid;
k) measuring the exit concentration of the ozone/oxygen admixture;
l) isolating the non-infectious biological fluid from the gas-fluid contacting device;
m) timing the duration that an ozone/oxygen admixture interfaces with the biological fluid in the gas-fluid contacting device;
n) compiling data including the inlet ozone concentration of the ozone/oxygen admixture, the flow rate and amount of the ozone/oxygen admixture, the flow rate and amount of the biological fluid, and the exit concentration of the ozone/oxygen admixture; and
o) calculating an amount of ozone delivered to the measured amount of the biological fluid and an amount of ozone absorbed by the measured amount of the biological fluid using the compiled data.

2. The method according to claim 1, wherein the biological fluid contains one or more mammalian blood products.

3. The method according to claim 1, wherein the gas-fluid contacting device includes fluid contacting surfaces.

4. The method according to claim 3, further comprising pre-treating the fluid contacting surfaces of the gas-fluid contacting device to prevent platelet adhesion and aggregation on the fluid contacting surfaces.

5. The method according to claim 3, further comprising pre-treating the fluid contacting surfaces of the gas-fluid contacting device with human serum albumin.

6. The method according to claim 2, wherein the virus is HIV.

7. The method according to claim 2 wherein the virus is hepatitis B.

8. The method according to claim 2, wherein the virus is hepatitis C.

9. The method according to claim 1, wherein the biological fluid is a synthetic fluid incorporating a substance with a biological origin.

10. The method according to claim 1 wherein the biological fluid is in an aqueous buffered carrier medium.

11. The method according to claim 10, wherein the aqueous buffered carrier medium is able to absorb ozone from the ozone/oxygen admixture.

12. The method according to claim 1, further comprising infusing a mammal with the biological fluid after the biological fluid has interfaced with the ozone/oxygen admixture.

13. The method according to claim 1, further comprising isolating the non-infectious biological fluid.

14. The method according to claim 1, wherein the gas-fluid contacting device includes gas-contacting surfaces constructed from ozone-inert construction materials.

15. The method according to claim 1, further comprising maintaining a temperature range of 4°-30° C. within the gas-fluid contacting device.

16. The method according to claim 1, further comprising treating the amount of biological fluid using one or more of a solvent-detergent, targeted is chemotherapeutics, photochemotherapeutics, gamma-irradiation, and photodynamic

17. The method according to claim 1, further comprising treating the amount of biological fluid using one or more additional viral inactivating processes.

18. The method according to claim 17, wherein the viral inactivating processes include a solvent-detergent, targeted chemotherapeutics, photochemotherapeutics, gamma-irradiation, and photodynamic antimicrobials.

19. The method according to claim 1, further comprising treating the non-infectious biological fluid using one or more additional viral inactivating processes.

20. The method according to claim 19, wherein the viral inactivating processes include a solvent-detergent, targeted chemotherapeutics, photochemotherapeutics, gamma-irradiation, and photodynamic antimicrobials.

21. A method of inactivating a virus in a biological fluid, comprising the steps of:
a) delivering a measured amount of an ozone/oxygen admixture to a measure amount of a biological fluid containing a virus;
b) interfacing the biological fluid with the ozone/oxygen admixture to generate byproducts of ozonation within the biological fluid as a result of at least a portion of the ozone/oxygen admixture being absorbed by the biological fluid, rendering the biological fluid non-infectious;
c) controlling the time during which the biological fluid interfaces with the ozone/oxygen admixture;
d) measuring the concentration of the ozone/oxygen admixture after the ozone/oxygen admixture has interfaced with the biological fluid containing the virus;
e) isolating the byproducts of ozonation within the non-infectious biological fluid; and
f) calculating an amount of ozone delivered to the measured amount of the biological fluid and an amount of ozone absorbed by the measured amount of the biological fluid.

22. A method according to claim 21, wherein the non-infectious biological fluid has maintained sufficient biological integrity.

23. The method according to claim 21, wherein the byproducts of ozonation includes lipid peroxides.

24. The method according to claim 21, wherein the biological fluid is an aqueous carrier medium.

25. The method according to claim 21, further comprising introducing a biocompatible agent that quenches the byproduct of ozonation.

26. The method according to claim 21, further comprising calculating the amount of ozone that was absorbed by the biological fluid.

27. The method according to claim 21, further comprising generating products of ozonation in an aqueous carrier medium which are subsequently added to a biological fluid.

28. The method according to claim 27, further comprising quantifying the amount of byproducts of ozonation generated so as to provide a correlation to the amount of ozone that was absorbed by the biological fluid.

29. A method of inactivating viruses, comprising the steps of:
a) manufacturing a measured amount of an ozone/oxygen admixture to be delivered to a gas-fluid contacting device such that an inlet concentration of the ozone/oxygen admixture is controlled;
b) measuring the inlet concentration of the ozone/oxygen admixture;
c) controlling and measuring the flow rate and the measured amount of the ozone/oxygen admixture to be delivered to the gas-fluid contacting device;
d) delivering the ozone/oxygen admixture to the gas-fluid contacting device;
e) selecting a biological fluid containing a virus to be delivered to the gas-fluid contacting device;
f) controlling and measuring the flow rate of the biological fluid to be delivered to the gas-fluid contacting device;
g) delivering a measured amount of the biological fluid to the gas-fluid contacting device;
h) interfacing an ozone/oxygen admixture at an inlet concentration with the biological fluid within the gas-fluid contacting device to;
1) generate byproducts of ozonation as a result of at least a portion of the inlet concentration of the ozone/oxygen admixture being absorbed by the biological fluid,
2) inactivate the virus, rendering the biological fluid non-infectious, while maintaining the biological integrity of the biological fluid;
i) controlling the time during which the biological fluid interfaces with the inlet concentration of the ozone/oxygen admixture;
j) releasing an ozone/oxygen admixture at an exit concentration from the gas-fluid contacting device after the ozone/oxygen admixture has interfaced with the biological fluid;
k) measuring the exit concentration of the ozone/oxygen admixture;
l) timing the duration that the ozone/oxygen admixture interfaces with the biological fluid in the gas-fluid contacting device;
m) compiling data including the inlet concentration of the ozone/oxygen admixture, the flow rate and the measured amount of the ozone/oxygen admixture, the flow rate and the measured amount of the biological fluid, and the exit concentration of the ozone/oxygen admixture,
n) calculating an amount of ozone delivered to the biological fluid and an amount of ozone absorbed by the biological fluid using the compiled data;
o) isolating the non-infectious biological fluid containing byproducts of ozonation;
p) introducing a biocompatible agent that quenches the byproducts of ozonation;
q) quantifying the amount of byproducts of ozonation generated so as to provide a correlation to the amount of ozone that was absorbed by the biological fluid.

30. A method of inactivating a virus in a biological fluid, comprising the steps of:
a) delivering a measured amount of an ozone/oxygen admixture to a measured amount of biological fluid containing a virus;
b) interfacing the biological fluid with the measured amount of the ozone/oxygen admixture resulting in at least a portion of the measured amount of the ozone/oxygen admixture being absorbed by the biological fluid, rendering the biological fluid non-infectious;
c) controlling the time during which the biological fluid interfaces with the measured amount of the ozone/oxygen admixture;
d) isolating the non-infectious biological fluid; and
e) calculating the amount of ozone absorbed by the fluid.

31. The method according to claim 30, further comprising maintaining the biological integrity of the biological fluid.

32. A method of inactivating a virus on a target surface, comprising the steps of:
a) delivering a measured amount of an ozone/oxygen admixture to a target surface infected with a virus;
b) interfacing the target surface with the measured amount of the ozone/oxygen admixture resulting in at least a portion of the measured amount of the ozone/oxygen admixture being absorbed by the target, rendering the target non-infectious;
c) controlling the time during which the target surface interfaces with the measured amount of the ozone/oxygen admixture;
d) isolating the non-infectious target surface; and
e) calculating the amount of ozone absorbed by the target surface.

33. The method according to claim 32, wherein the non-infectious target surface has sufficient biological integrity.
